(19) United States
(12) Patent Application Publication
(10) Pub. No.: US 2008/0058738 A1
(43) Pub. Date: Mar. 6, 2008

(54) Derivatized expanded starch for odor control

(75) Inventors: Frank Grosch Roberts, Glencoe, IL (US); Corey Thomas Cunningham, Larsen, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/513,587

(22) Filed: Aug. 31, 2006

(51) Int. Cl. A61F 13/15

(52) U.S. Cl. 604/359; 604/360

(57) ABSTRACT

An absorbent article includes a plurality of derivatized expanded starch particles to control odor. The derivatized expanded starch particles include an expanded starch particle base and at least one transition metal chemically bonded to the expanded starch particle base. The transition metal is coordinatively bonded to a bridging compound and the bridging compound is covalently bonded or physically absorbed to a surface of the expanded starch particle bases.

### BACKGROUND OF THE INVENTION

The control of unpleasant odors has long been a goal of various products on the market such as, for example, infant diapers, adult incontinence products, feminine products, training pants, pet litters, and the like. As such, many attempts have been made to formulate an effective odor removal system and the various products currently available provide varying degrees of odor control.

Some products are designed to cover up odors by emitting stronger, more dominant odors, for example scented air freshener sprays and candles. Other products are designed to combat odorous compounds, including ammonia, methyl mercaptan, trimethylamine, and other odiferous sulfides and amines commonly found in soiled absorbent articles, by removing these compounds from a medium by deodorizing agents adapted to absorb these compounds.

In general, odors may be controlled by various means such as, for example, physical adsorption, chemical adsorption, malodor prevention, odor modification, and the like, and combinations thereof. In physical adsorption, malodorous molecules are attracted by weak physiochemical forces (i.e., non-specific interactions) including dipole-dipole and van der Waals forces. As such, a material with a high surface area, such as activated carbon, will generally bind more malodorous compounds than materials having a smaller surface area. For example, activated charcoal and sodium bicarbonate have commonly been used to absorb odors.

In chemical adsorption (i.e., chemisorption), malodorous molecules are attracted and strongly bound by chemical bonds. Generally, chemical adsorption creates stronger bonds than physical adsorption. The bonds created may be considered permanent or semi-permanent. Additionally, chemical adsorption generally requires one or more specific active sites for bonding.

Malodors may also be eliminated or inhibited by eliminating or inhibiting their production. Various methods for achieving this result include inhibiting enzymes that facilitate the formation of a malodor or killing the bacteria that produces such enzymes. For example, ammonia, a major component of urine odor, is produced through the hydrolysis of urea. By inhibiting urease, such as by modifying the pH of the environment, the first step to generating ammonia is inhibited.

Odors may also be controlled via odor modification which may include masking, blocking and/or complimenting the target odor or odors. Masking does not eliminate malodors but diminishes the perception of the odor by overwhelming the olfactory nerves with a pleasant fragrance. However, because the malodor is not eliminated, the malodor may still be detected or the masking odor may unpleasantly mix with the malodor. Likewise, blocking does not eliminate or trap malodors but works on the principle that the olfactory nerves can be blocked by a molecule with a similar topography as the target malodor. The similar molecule may be less offensive or undetectable by the human nose and therefore block the olfactory nerves that would normally bind with the malodorous compound. By blocking the olfactory nerves, the malodor remains undetected. Likewise, complimenting does not eliminate or trap the malodor but works on the principle that the malodor can be combined with the other compounds to make a pleasant smell. For example, skatole and indole are the major components of sewer odor. They are also, however, components of the oil of jasmine. By adding the missing components of jasmine's fragrance to skatole and indole a pleasant fragrance may be created that incorporates the malodor into a pleasant smell.

Many attempts have also been made to minimize or eliminate odors after disposal of odiferous products. For example, diaper pails with tight fitting lids have long been used in an attempt to contain diaper odors. However, when opened, the odors are allowed to escape. Similarly, methods and apparatus for individually sealing absorbent articles in plastic tube-like pouches have been proposed to minimize odor escape. However, this does not provide a solution for disposal away from home. Finally, disposal bags have been suggested for portability but most only provide partial containment and some masking.

Therefore, in spite of these previous odor control efforts, there remains a need for odor control particles to reduce, eliminate, and/or mask malodors when used alone or when incorporated in various products.

### SUMMARY OF THE INVENTION

In one aspect, the present invention provides an absorbent article with a plurality of derivatized expanded starch particles. The derivatized expanded starch particles include an expanded starch particle base and at least one transition metal chemically bonded to the expanded starch particle base. In some embodiments, the transition metal is copper, iron, manganese, zinc or silver. In some embodiments, the transition metal includes at least one ligand chemically bonded thereto, wherein the ligand is citronellol, benzaldehyde, alpha-pinene, 3-methylbutyl acetate, cymene, menthol, limonene or 2-butanone. In some embodiments, the transition metal is coordinatively bonded to a bridging compound and the bridging compound is covalently bonded to a surface of the expanded starch particle bases. In some embodiments, the bridging compound is a siloxane anchoring group covalently bonded to a metal binding site. In some embodiments, the transition metal is coordinatively bonded to a bridging compound and the bridging compound is physically absorbed to a surface of the expanded starch particle bases. In some embodiments, the bridging compound includes an ionic anchoring group covalently bonded to a metal binding site, wherein the ionic anchoring group is selected from the group consisting of quarternary ammonium, carboxylate, sulfonate, phosphate, sulfate and phosphonate.

In some embodiments, at least one first derivatized expanded starch particle includes a first expanded starch particle base, a first transition metal chemically bonded to the first expanded starch particle base, and a second transition metal chemically bonded to the first expanded starch particle base, wherein the first transition metal and the second transition metal are different. In some embodiments, at least one first derivatized expanded starch particle includes a first expanded starch particle base having a first transition metal chemically bonded to the first expanded starch particle base and at least one second derivatized expanded starch particle includes a second expanded starch particle base having a second transition metal chemically bonded to the second expanded starch particle base, wherein the first and second transition metals are different. In some embodiments, the first transition metal is copper and the second transition metal is iron.

In another aspect, an absorbent article includes at least one nonderivatized expanded starch particle and at least one derivatized expanded starch particle, wherein the at least one derivatized expanded starch particle includes an expanded starch particle base and one or more transition metals chemically bonded to the expanded starch particle base. In some embodiments, the transition metal is copper, iron, manganese, zinc or silver. In some embodiments, the transition metal includes at least one ligand chemically bonded thereto, wherein the ligand is citronellol, benzaldehyde, alpha-pinene, 3-methylbutyl acetate, cymene, menthol, limonene or 2-butanone. In some embodiments, the transition metal is chemically bonded to a bridging compound and the bridging compound is covalently bonded to a surface of the expanded starch particle bases. In some embodiments, at least one first derivatized expanded starch particle includes a first expanded starch particle base, a first transition metal chemically bonded to the first expanded starch particle base, and a second transition metal chemically bonded to the first expanded starch particle base, wherein the first transition metal and the second transition metal are different. In some embodiments, at least one first derivatized expanded starch particle includes a first expanded starch particle base having a first transition metal chemically bonded to the first expanded starch particle base and at least one second derivatized expanded starch particle having a second expanded starch particle base having a second transition metal chemically bonded to the second expanded starch particle base, wherein the first and second transition metals are different.

In another aspect, an absorbent article includes a plurality of derivatized expanded starch particles, a first transition metal, and a second transition metal, wherein the first and second transition metals are different. In some embodiments the derivatized expanded starch particles include an expanded starch particle base and the first transition metal and the second transition metal are chemically bonded to bridging compounds and the bridging compounds are covalently bonded to a surface of the expanded starch particle bases. In some embodiments, at least one first derivatized expanded starch particle includes a first expanded starch particle base, wherein the first transition metal is chemically bonded to the first expanded starch particle base, and the second transition metal is chemically bonded to the first expanded starch particle base. In some embodiments, at least one first derivatized expanded starch particle includes a first expanded starch particle base having the first transition metal chemically bonded to the first expanded starch particle base and at least one second derivatized expanded starch particle includes a second expanded starch particle base having the second transition metal chemically bonded to the second expanded starch particle base.

### BRIEF DESCRIPTION OF THE DRAWINGS

FIG. 1 representatively illustrates the results of GC-Headspace testing for various test materials against dimethyldisulfide.

FIG. 2 representatively illustrates the results of GC-Headspace testing of expanded starch exposed to three different conditions against dimethyldisulfide.

FIG. 3 representatively illustrates the results of GC-Headspace testing for expanded starch relative to three different malodors.

### DETAILED DESCRIPTION

Reference now will be made in detail to various embodiments of the invention, one or more examples of which are set forth below. Each example is provided by way of explanation, not as a limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

The present invention relates to odor control. Specifically, the present invention relates to non-derivatized expanded starches and metal derivatized expanded starches (collectively “expanded starches”) that are useful in controlling gaseous compounds and/or odorous compounds.

As used herein, the term “odorous compound” or “odor” refers to any molecule or compound detectable to the olfactory system. Odorous compounds can exist as a gaseous compound and can also be present in other media such as liquids. “Gaseous compound” or “gas” refers to any molecule or compound that can exist as a gas or vapor. Examples of odorous compounds include mercaptans (e.g., ethyl mercaptan), alcohols (e.g., hexanol), amines (e.g., ammonia, trimethylamine (TMA), triethylamine (TEA), etc.), sulfides (e.g., hydrogen sulfide, dimethyl disulfide (DMDS), etc.), ketones (e.g., 2-butanone, 2-pentanone, 4-heptanone, etc.), carboxylic acids (e.g., isovaleric acid, acetic acid, propionic acid, etc.), aldehydes (e.g., heptanal), terpenoids and imines (e.g., pyridine).

The major odorous components of common household odors, such as cat odor, dog odor, garbage odor, body odor, foot odor, food odor, urine odor, feces odor and tobacco odor are amines, sulfur compounds, carboxylic acids and aldehydes. For example, the generation of odor from urine is mostly based on chemical and biological degradation of urine components resulting in amines, ammonia, methyl mercaptan and hydrogen sulfide, for example. Similar odorants can also be found in feces odor and body odor. Additionally, enzymes such as urease can convert urea, a major component in urine, to ammonia and thereby increase the generation of odors in urine. Aliphatic acids such as valeric, isovaleric, butyric and acetic acids are commonly found to be the major odor components in body odors, foot odor, tobacco smoke, raw meat, garbage (kitchen) odor, cat odor, and the musty smell of basements and cellars.

The present invention utilizes expanded starches to control odors. In some embodiments, the present invention utilizes expanded starches to control odors in absorbent articles. As used herein, an “absorbent article” refers to any article capable of absorbing water or other fluids. Examples of some absorbent articles include, but are not limited to, personal care absorbent articles, such as diapers, training pants, absorbent underpants, adult incontinence products, feminine hygiene products (e.g., sanitary napkins), swim wear, baby wipes, and the like; medical absorbent articles, such as garments, fenestration materials, underpads, bandages, absorbent drapes and medical wipes; food service wipers; clothing articles; and the like. Materials and processes suitable for forming such absorbent articles are well known to those skilled in the art.

Expanded starch is safe, white and has a relatively low cost as compared with other odor control agents. As disclosed in WO 2005/011836 A1 to Clark et al. (Clark), which is incorporated herein by reference where not contradictory, starch is a biopolymer produced from plants and is composed of a mixture of amylase and amylopectin, which have α-linkages. While “native” starch has a surface area of about 1 m²/g, “expanded” starch can achieve surface areas of about 200 m²/g or greater. Therefore, the expanded starch of the present invention may have a surface area of at least 20 m²/g, at least 30 m²/g, at least 50 m²/g, at least 75 m²/g, at least 100 m²/g, at least 200 m²/g or at least 220 m²/g. Surface area may be determined by the physical gas adsorption (B.E.T.) method of Bruanauer, Emmet, and Teller, as described in the Journal of American Chemical Society, Vol. 60, 1938, p. 309, with nitrogen as the adsorption gas. Whereas, Clark discloses the use of high surface area polysaccharides for separating chemical compounds (e.g., chromatography), the high surface area of the expanded starches of the present invention have been found useful for absorbing gaseous compounds and/or odorous compounds from the surrounding environment.

Clark also discloses that the higher the amylase percent the larger the surface area of the expanded starch. Therefore, the expanded starch of the present invention may have an amylase content of at least 20%, at least 30%, at least 40%, at least 50%, or at least 60%. Suitable high amylase starches include corn starch, such as HYLON VII brand high amylase starch available from National Starch and Chemical having offices in Bridgewater, N.J. Other suitable starches include, for example, potato starch, wheat starch, rice starch, and the like, and combinations thereof.

One suitable method for preparing high surface area starches is disclosed in Clark and includes the general steps of i) gelatinizing the starch in the presence of water to create a starch and water gel; ii) allowing the starch to retrograde; and iii) exchanging the water in the retrograded starch gel with a water-miscible non-solvent for starch which has a lower surface tension than water, such as ethanol.

Specifically, the expanded starch of the present invention was prepared as follows. First, 37.5 grams of native corn starch (HYLON VII brand food starch available from National Starch and Chemical having offices in Bridgewater, N.J.) was placed into a 1000 ml heat tolerant flask (PYREX brand) with a screw cap. Next, 750 ml of Millipore filtered water (0.22 μm) was added to the flask. The mixture was heated in an oven at 130° C. for 48 hours. The resultant gel was allowed to return to room temperature and then placed in a cooler at 3° C. for five days. Ice that formed was allowed to melt and the water was filtered off using vacuum filtration. The resultant semi-solid aqua-gel was successively equilibrated by stirring with 25, 50, 75, and 100% aqueous ethanol (about 75 ml each). After each equilibration, the liquid was filtered off using vacuum filtrations. The solid was then filtered to remove most of the water without letting the solid dry. It was then equilibrated with 100% ethanol to dehydrate the solid. The suspension was dried for 15 hours in a vacuum oven at 50° C. to obtain a free-flowing powder.

The expanded starch of the present invention was compared with other materials with regard to odor adsorption. Odor adsorption was evaluated using headspace gas chromatography (GC-Headspace) techniques. The GC-Headspace procedure measured the amount of an odoriferous compound remaining in the gas phase after exposure to various test materials. The GC-Headspace testing was conducted with an Agilent Technologies 7694 headspace sampler interfaced with an Agilent Technologies 6890 gas chromatograph which was equipped with a flame ionization detector (FID) (Agilent Technologies, Waldbronn, Germany).

Helium was used as the carrier gas and Table 1 details the operating conditions. For triethylamine (TEA), trimethylamine (TMA), dimethyldisulphide (DMDS) and ethyl mercaptan, a DB-624 column (J&W Scientific, Inc. of Folsom, Calif.) having a length of 30 meters, an internal diameter of 0.25 millimeters, and a 1.4-micron film was used.

TABLE 1
GC-Headspace Conditions

| General | | Oven Program | |
|---|---|---|---|
| Oven | 100° C. | Cryogenics | Off |
| Loop | 125° C. | Initial Temp | 40° C. |
| Transfer Line | 125° C. | Initial Hold | 5.0 min |
| Vial Equilibration | 5.0 min | Equilibration | 3.0 min |
| Time | | Time | |
| Pressurization Time | 0.2 min | Total Run Time | 9.4 min |
| Loop Fill Time | 0.2 min | Ramp | 25° C./min to |
| Loop Equilibration | 0.1 min | | 125° C., hold for |
| Time | | | 1.0 min |
| Injection Time | 0.1 min | | |

A typical test procedure included placing about 0.14 grams of the test material inside a 20-cubic centimeter headspace vial. The amount of sample can be adjusted to keep the measurement within range of the instrument for better accuracy. Using a syringe, an aliquot of an odoriferous agent was also placed in the vial, taking care not to let the liquid and test material contact. The vial was then sealed with a cap and septum and placed in the GC-Headspace oven. After ten minutes, a hollow needle was inserted through the septum and into the vial to extract a 1-cubic centimeter sample of the headspace (air inside the vial). The sample was then transferred into the GC-Headspace chromatograph. Initially, a control vial with only the aliquot of odoriferous agent (no test material) was tested to define the maximum concentration of odoriferous agent. To calculate the amount of odoriferous agent removed from the headspace by the test material, the peak area for the odoriferous agent from the vials collected in the presence of the test materials were compared to the peak area from the odoriferous agent control vial (no test material).

GC-Headspace chromatography testing as described above was performed on several materials (test materials), as listed in Table 2 below, that purportedly control odors.

TABLE 2
GC test materials

| Label | Description |
|---|---|
| Blank | Signal area measured with no test material included to define the maximum concentration of the |
| | odiferous agent |
| Citra-Max | Signal area measured with Citra-Max Fresh ® brand pet litter as the test material; Citra-Max Fresh ® |
| | brand pet litter is an all natural citrus litter available from Meow911.com Inc. having offices in Mission |
| | Viejo, California |
| Starch | Signal area measured with native corn starch as the test material; the native corn starch was obtained |
| | from National Starch and Chemical sold under the brand name HYLON VII |
| Swheat Scoop | Signal area measured with Swheat Scoop ® brand litter as the test material; Swheat Scoop ® brand |
| | litter is made from naturally processed wheat and is available from Pet Care Systems (PCS) located in |
| | Detroit Lakes, Minnesota |
| Odorzout | Signal area measured with ODORZOUT ® brand product as the test material; ODORZOUT ® brand |
| | product is a blend of natural zeolite minerals mined and produced in Arizona and is available from |
| | No Stink, Inc. having offices in Phoenix, Arizona |
| Everclean | Signal area measured with Ever Clean ® brand pet litter as the test material; Ever Clean ® brand litter is |
| | made of natural minerals and clays and is manufactured for the Clorox Pet Products Company having |
| | offices in Oakland, California |
| Exquisicat | Signal area measured with Exquisicat Crystals ® brand pet litter as the test material; Exquisicat |
| (Grey) | Crystals ® brand litter is 100% silica sand and is distributed by Pacific Coast Distributing, Inc. having |
| | offices in Phoenix, Arizona |
| Nature's Miracle | Signal area measured with Nature's Miracle ® brand pet litter as the test material; Nature's Miracle ® |
| | brand pet litter is made of corncob granules having natural enzymes and is available from Pets ‘N |
| | People, Inc., which is a subsidiary of Eight in One Pet Products having offices in Hauppauge, New York |
| World's Best | Signal area measured with World's Best Cat Litter ™ brand pet litter as the test material; World's Best |
| | Cat Litter ™ brand litter is made from whole kernel corn and is available from GPC Pet Products, a |
| | division of Grain Processing Corp. having offices in Muscatine, lowa |
| Exquisicat | Signal area measured with Exquisicat ® brand pet litter as the test material; Exquisicat ® brand litter is |
| | made of natural zeolites and is distributed by Pacific Coast Distributing, Inc. having offices in Phoenix, |
| | Arizona |
| Nanoparticles | Signal area measured with iron coated silica nanoparticles as the test material; the nanoparticles are |
| | described in U.S. Pat. No. 2005/0084438 |
| Expanded | Signal area measured with expanded corn starch as the test material; the native corn starch was |
| Starch | obtained from National Starch and Chemical sold under the brand name HYLON VII and was expanded |
| | according to the method described herein |

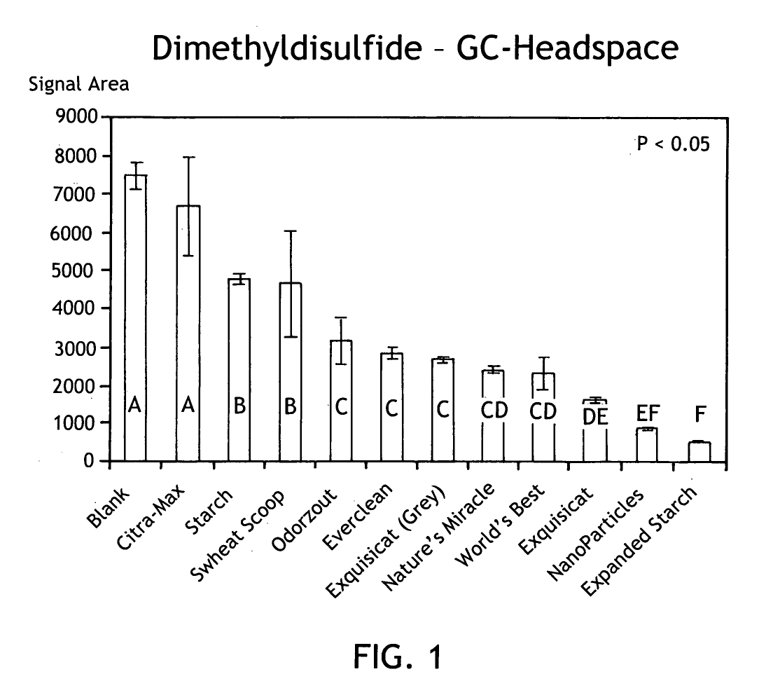

The test materials were evaluated using dimethyldisulfide as the odiferous agent. Dimethyldisulfide was chosen as the test odorant because it is a primary odorant in urine, feces and menses. As such, many disposable absorbent products would benefit from a reduction in dimethyldisulfide. The results of this testing are summarized in Table 3 below and graphically illustrated in FIG. 1.

TABLE 3
Removal of Dimethyldisulfide

| | Approximate | Approximate |
|---|---|---|
| | Signal Area | reduction as |
| Test Material | μV · s (thousands) | compared to blank |
| Blank | 7500 | — |
| Citra-Max | 6700 | 10% |
| Starch | 4700 | 37% |
| Swheat Scoop | 4500 | 40% |
| Odorzout | 3100 | 60% |
| Everclean | 3100 | 60% |
| Exquisicat (Grey) | 2700 | 64% |
| Nature's Miracle | 2400 | 68% |
| World's Best | 2400 | 68% |
| Exquisicat | 1700 | 77% |
| Nanoparticles | 900 | 88% |
| Expanded Starch | 600 | 92% |

Referring now to FIG. 1 and Table 3, the reported signal area is indicative of the relative adsorption of the various test materials because the signal area is proportional to the molecular concentration of the odorant in the vial. Therefore, the lower the signal area, the higher the adsorption by the test material. The blank test represents the maximum concentration of the odorant because no test material was present to absorb any of the odorant.

The various test materials showed a range of odor adsorption from about a 10% reduction to about a 92% reduction as compared to the blank. The expanded starch of the present invention demonstrated the greatest reduction by absorbing about 92% of the odorant in this test. A comparison of the native starch signal area to the expanded starch signal area shows a reduction in signal area by about 87% (i.e., from about 4700 μV·s (thousands) to about 600 μV·s (thousands)). In other words, the expanded starch absorbed about 92% of the odorant as compared to 37% by the native starch. It is believed that the greatly expanded surface area of the expanded starch contributed to this result by providing more available locations for the odorant to bond. Therefore, more odor molecules were absorbed from the headspace.

By way of comparison, the iron coated nanoparticles reduced the concentration of odorant by about 88% as compared to the blank. These results suggest that the expanded starch of the present invention may be a suitable alternative to the nanoparticles as regarding general odor absorption. Additionally, the expanded starch achieved these levels without the use of metals on the surface of the odor control material. This may be advantageous in certain embodiments.

Likewise, the expanded starch of the present invention may be a suitable alternative to some of the “natural” odor absorbers tested. For example, Swheat Scoop® brand litter is made from naturally processed wheat and reduced the test odor by about 40%. The World's Best Cat Litter™ brand litter is made from whole kernel corn and reduced the test odor by about 68%. By comparison, the expanded starch of the present invention is made from corn starch and reduced the test odor by about 92%.

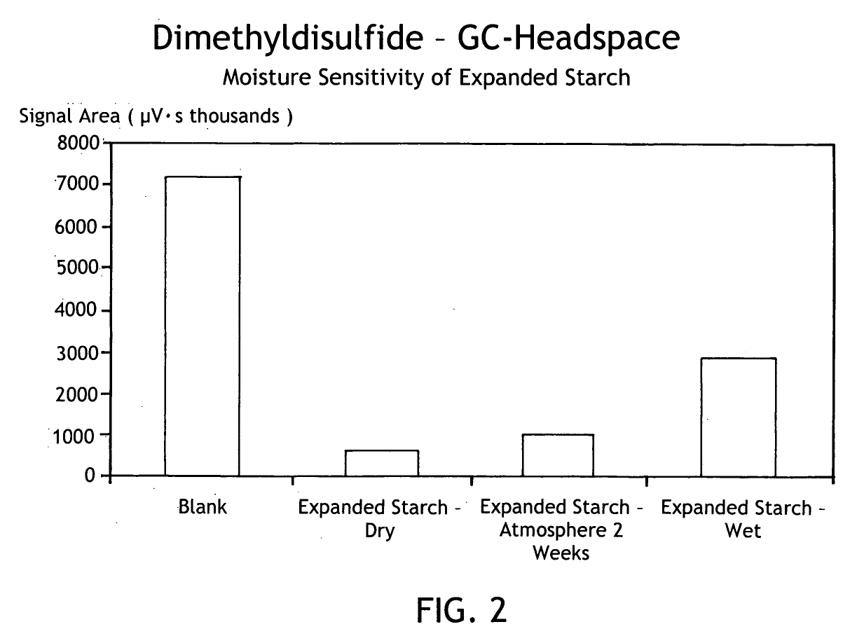

As previously discussed, the increased surface area of the expanded starch is believed to result in the high odorant adsorption observed. However, the expanded surface area of the starch may partially collapse if wetted thereby resulting in a loss of surface area. As such, the adsorptive characteristics of the expanded starch may be reduced because of water molecules occupying some or all of the adsorption sites on the expanded starch through hydrogen bonding. To study the effects of environmental moisture, a sample of dry expanded starch was exposed to the atmosphere for 2 weeks. Similarly, to study the effects of liquid contact with the expanded starch, a sample of dry expanded starch was mixed with water. Both samples and a dry control were evaluated relative to a blank using the GC-Headspace gas chromatography procedure described herein using dimethyldisulfide as the odorant. The results are summarized in Table 4 below and graphically illustrated in FIG. 2.

TABLE 4
Removal of Dimethyldisulfide

| | Approximate | Approximate |
|---|---|---|
| | Signal Area | reduction as |
| Test Material | μV · s (thousands) | compared to blank |
| Blank | 7100 | — |
| Dry Expanded Starch | 550 | 93% |
| Environmentally | | |
| Exposed Starch | 990 | 86% |
| (2 weeks) | | |
| Wet Expanded Starch | 2800 | 60% |

As can be seen in the data of table 4, the dry expanded starch reduced the signal area by about 93% as compared to the blank (i.e., from about 7100 μV·s (thousands) to about 550 μV·s (thousands)). By comparison, the sample exposed to the atmosphere for 2 weeks reduced the signal area by about 86% as compared to the blank (i.e., from about 7100 μV·s (thousands) to about 990 μV·s (thousands)). Therefore, the exposure to the atmosphere appears to have reduced the odor absorbent capacity of the expanded starch by about 7%. Further by comparison, the sample of dry expanded starch mixed with water reduced the signal area by about 60% as compared to the blank (i.e., from about 7100 μV·s (thousands) to about 2800 μV·s (thousands). Therefore, exposure to water appears to have reduced the capacity of the expanded starch to absorb the odorant by about 33% as compared to the dry sample and about 26% as compared to the atmospherically exposed sample.

As such, the expanded starches (derivatized, non-derivatized, and combinations thereof) of the present invention may be protected from humidity and/or moisture in various embodiments. The expanded starches of the present invention may also be protected from various odors in some embodiments. For example, to increase shelf life, the expanded starches of the present invention may be located, at least partially in water impermeable and/or vapor impermeable enclosures. In some embodiments, the expanded starches may be located, at least partially, in one or more water impermeable and vapor permeable enclosures.

In some embodiments, the expanded starches may be located, at least partially, in one or more water impermeable and vapor impermeable enclosures that are adapted to be transitioned to water impermeable and vapor permeable enclosures. For example, the expanded starches of the present invention may be located within an air tight enclosure. The enclosure may include one or more openings sized to allow vapor molecules to pass but prevent water molecules from passing. The openings may be covered with an air tight seal to prevent vapor from passing until removed. The seal may take any suitable form, such as, for example, a piece of adhesive tape. In some embodiments, the enclosure may be adapted such that the seal can be removed thereby exposing the openings and rendering the enclosure liquid impermeable and vapor permeable.

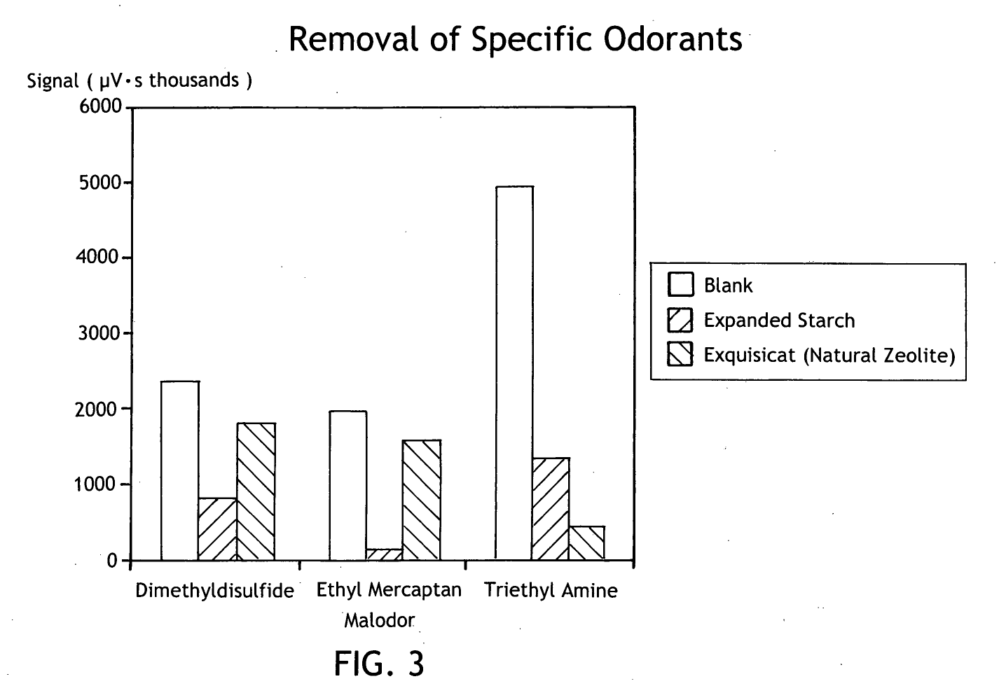

To evaluate the odor absorption of the present invention relative to specific odorants, the GC-Headspace testing, as described herein, was performed using dimethyldisulfide, ethyl mercaptan, and triethylamine (TEA) as test odorants relative to a blank sample. As discussed previously, these odorants are particularly relevant to disposable personal care items such as diapers, training pants, feminine hygiene products, and the like. The codes tested included the expanded starch of the present invention, and the Exquisicat® brand natural zeolite cat litter. The results of this evaluation are summarized in Table 5 below and are graphically illustrated in FIG. 3.

TABLE 5
Removal of Specific Odorants

| | | Approximate | |
|---|---|---|---|
| | | Signal Area | Approximate |
| | | μV · s | reduction as |
| Odorant | Test Material | (thousands) | compared to blank |
| Dimethyldisulfide | Blank | 2300 | — |
| | Expanded Starch | 800 | 65% |
| | Exquisicat | 1800 | 22% |
| Ethyl Mercaptan | Blank | 1950 | — |
| | Expanded Starch | 125 | 94% |
| | Exquisicat | 1350 | 31% |
| Triethylamine | Blank | 4900 | — |
| | Expanded Starch | 1300 | 73% |
| | Exquisicat | 450 | 91% |

Referring now to FIG. 3 and Table 5, the expanded starch of the present invention reduced the signal strength of the various odorants as compared to the blank. Specifically, the expanded starch particles of the present invention reduced the concentration of dimethyldisulfide by about 65%, ethyl mercaptan by about 94% and triethylamine by about 73%. As compared to Exquisicat® brand cat litter (with natural zeolites); the expanded starch of the present invention had a greater reduction in concentration for dimethyldisulfide (i.e., 65% versus 22%) and ethyl mercaptan (i.e., 94% versus 31%). However, for triethylamine, the expanded starch did not reduce the concentration of the odorant to the same extent as Exquisicat® brand cat litter (with natural zeolites) (i.e., 73% versus 91%). Based on this examination, the expanded starch demonstrated effectiveness for absorbing these odors. These results also suggest that the surfaces of the expanded starch and the natural zeolite may have different affinities to different odorants. Therefore, combinations of various odor control substances may be effective for reducing a spectrum of odors.

In some embodiments, the non-derivatized expanded starch particles may be treated with an antimicrobial agent to prevent the formation of odors due to the metabolic processes of enzymes in organisms. The expanded starch particles may contact an aqueous or non-aqueous solution of the desired antimicrobial agent for a set amount of time. During this time, the antimicrobial agent would be expected to physically adsorb onto the expanded starch material thereby creating an antimicrobial expanded starch. The antimicrobial agent could be any one of the FDA and EPA recognized antimicrobials or cosmetic preservatives as well as a botanical extract shown to have antimicrobial efficacy. Specific examples include, but are not limited to, benzalkonium chloride and other salts thereof, benzethonium chloride and other salts thereof, methylbenzethonium chloride and other salts thereof, povidone-iodine, boric acid, chlorhexidine digluconate and other salts thereof, triclosan, polyhexamethylene biguanide hydrochloride and other salts thereof, citric acid, 2-bromo-2-nitro-1,3-propanediol, parabens, chlorphenesin, methylisothiazolinone, to name a few. The antimicrobial expanded starch particles would be expected to provide odor adsorption similar to that described for the unmodified expanded starch particles. In most cases, the antimicrobial expanded starch would be expected to remain white with the exception of povidone-iodine. In addition to the odor adsorption ability, the antimicrobial expanded starch would also be expected to control odors produced by microorganisms during extended exposure to insults because the antimicrobial may reduce or eliminate the microorganisms.

The expanded starches described herein may also be derivatized to form a coordinate complex with one or more transition metals. The transition metals present on the surface of the expanded starch particle bases of the present invention are believed to provide one or more active sites for capturing and/or neutralizing odorous compounds (i.e., specific chemical adsorption). The active sites may be free, or may include one or more ligands bonded weakly enough so that they are replaced by an odorous molecule when contacted therewith. Additionally, the expanded starch particle base of the derivatized expanded starch particles are believed to still have a large surface area that is useful in absorbing other odorous compounds as described above (i.e., general physical adsorption).

As used herein, the term “transition metal” refers to metals located in the d-block of the periodic table of elements such as, for example, scandium, titanium, vanadium, chromium, manganese, iron, cobalt, nickel, copper, zinc, silver and gold.

The derivatized expanded starch of the present invention includes an expanded starch particle as the base structure. Bonded to the expanded starch particle base may be a bridging structure to which is attached a transition metal. The transition metal may have one or more ligands attached thereto.

Expanded starch provides a good material to use as the base for the coordinated metal complex because it allows for easy synthetic manipulation, has very few side reactions, is relatively inexpensive, biodegradable and renewable. Additionally, as a base material for odor control, it is a good starting material because it gives a high degree of generic odor adsorption on its own due to the expanded surface of the starch as discussed previously. When modified with a specific metal, the expanded starch may “target” the adsorption of specific types of odors in addition to the generalized absorption achieved through the expanded surface. Additionally, expanded starch is white and is therefore well received in consumer products. It will remain white during the synthetic manipulation up until the metal atom is added. The metal atom can affect color, especially at higher loadings, but with proper choice of the metal and the binding site, the color can be controlled and utilized to complement the design of the finished product.

In some embodiments, the present invention controls odors, at least in part, via transition metals, such as scandium, titanium, vanadium, chromium, manganese, iron, cobalt, nickel, copper, zinc, silver, gold, etc. Single metallic, as well as dimeric, trinuclear and cluster systems may be used. Without being limited by theory, it is believed that the transition metal provides one or more active sites for capturing and/or neutralizing an odorous compound. For example, the transition metal may be effective in removing odorous compounds, such as mercaptans (e.g., ethyl mercaptan), ammonia, amines (e.g., trimethylamine (TMA), triethylamine (TEA), etc.), sulfides (e.g., hydrogen sulfide, dimethyldisulfide (DMDS), etc.), ketones (e.g., 2-butanone, 2-pentanone, 4-heptanone, etc.), carboxylic acids (e.g., isovaleric acid, acetic acid, propionic acid, etc.), aldehydes, terpenoids, hexanol, heptanal, pyridine, and combinations thereof.

If desired, more than one type of transition metal may be utilized. This has an advantage in that certain metals may be better at removing specific odorous compounds than other metals. In other words, specific odors may be “targeted” by selecting specific transition metals. For example, copper may be more effective in removing sulfur and amine odors, manganese may be more effective in removing carboxylic acids and/or aldehydes, iron may be more effective in removing amines because of a strong affinity for amines, silver may be more effective in removing aldehydes because of a high affinity for aldehydes, and zinc may be effective in removing various odiferous compounds because it has a general affinity for all odiferous compounds.

The transition metals may be incorporated onto the surface of the expanded starch particle bases in a variety of ways. For instance, expanded starch particles may simply be mixed with a solution containing the appropriate transition metal in the form of a salt, such as those containing a copper(II) ion (Cu²⁺), silver(I) ion (Ag⁺), gold(I) and (III) ion (Au⁺ and Au³⁺), iron(II) ion (Fe²⁺), iron(III) ion (Fe³⁺), and so forth. Such solutions are generally made by dissolving a metallic compound in a solvent resulting in free metal ions in the solution. Generally, the metal ions are drawn to and adsorbed onto the expanded starch particle bases due to their electric potential differences, i.e., they form an “ionic” bond.

In many instances, however, it is desired to further increase the strength of the bond formed between the metal and expanded starch particle bases, e.g., to form a “coordinate” and/or “covalent bond.” Although ionic bonding may still occur, the presence of coordinate or covalent bonding may have a variety of benefits, such as reducing the likelihood that any of the metal will remain free during use (e.g., after washing). Further, a strong adherence of the metal to the expanded starch particle bases is also believed to optimize odor adsorption effectiveness. As used herein, a “coordinate bond” refers to a shared pair of electrons between two atoms, wherein one atom supplies both electrons to the pair. As used herein, a “covalent bond” refers to a shared pair of electrons between two atoms, wherein each atom supplies one electron to the pair.

In some embodiments, a “bridging compound” may be employed to provide an anchoring site to the expanded starch base and a binding site for the transition metal. These bridging compounds could be either small molecules or macromolecules. A small molecule would be of a limited number of atoms and have discrete anchoring and metal binding sites. A macromolecular bridging compound would be much larger in nature, possessing multiple anchoring sites that may sometimes be ionizable when dissolved in a suitable solvent (e.g., water, alcohols, etc.). These macromolecular compounds may be, for instance, polymers, hyperbranched polymers, dendrimers, oligomers, etc.

The macromolecular bridging compounds may contain one or more anchoring sites that are positively charged (cationic), negatively charged (anionic) and/or neutral. For example, the bridging compounds may be physically absorbed to the surface of the expanded starch particle bases. The bridging compound may include an ionic anchoring group covalently bonded to a metal binding site, such as, for example, quarternary ammonium cation, carboxylate anion, sulfonate anion, phosphate anion, sulfate anion and phosphonate anion.

In some embodiments, water-soluble bridging compounds having one or more basic anchoring sites, such as amine or imine ligands, may be used. For instance, examples of suitable basic reactive anchor site-containing bridging compounds may include, but are not limited to, polylysine, polyvinylamine, polyallylamine, polyalkylimine, etc. Polyalkylimines, for example, are water-soluble, hydrophilic, polyamines evolved from aziridine and azetidine monomers, such as 1-unsubstituted imines, 1-substituted basic imines, activated imines (1-acyl substituted imines), isomeric oxazolines/oxazines, and so forth. Polyalkylimines may be linear or highly branched, thereby possessing primary, secondary and tertiary amine groups. In one particular embodiment, the polyalkylimine is polyethyleneimine, which can be either linear or branched. Linear polyethyleneimine may be prepared via hydrolysis of poly(2-ethyl-2-oxazoline), while branched polyethyleneimine may be prepared by cationic chain-growth polymerization, either alone or with other monomers suitable for copolymerization with ethyleneimine. Other suitable bridging compounds are disclosed in U.S. 2005/0084474 to Wu et al. published Apr. 21, 2005, the entirety of which is incorporated herein by reference where not contradictory.

In one embodiment, the bridging compound utilizes a siloxane anchoring group covalently bonded to the metal binding site. The siloxane anchoring group could be a triethoxy-, trimethoxy-, trichloro-, or the like. The siloxane additionally contains a hydrocarbon functional group such as propylamine, propylmercaptan, propylurea, para-aniline, propylisocyanate, propylethylenediamine, or the like. In these embodiments, the siloxane anchoring group may first be covalently attached to the starch surface. The remaining functional group on the bridging compound may then be utilized as the metal binding site “as is,” such as, for example, in the case of propylurea or propylethylenediamine, or may be further reacted to create a metal binding site, such as, for example, in the case of propylamine or propylisocyanate. In addition to utilizing siloxane anchoring groups, other reactive silicone-based anchoring groups may be utilized such as for example, triethoxysilane, trichlorosilane, PSS-(2-(3,4-epoxycyclohexyl)ethyl)-heptaisobutyl substituted, PSS-(hydridodimethylsilyloxy)-heptacyclopentyl substituted, or the like.

Additionally, creating coordinate bonds with bridging structures may reduce steric hindrance. Steric hindrance refers to the ‘molecular congestion’ around either the binding site or the active site. If the binding site is crowded by the molecular structure of the starch, the metal will not be able to enter the binding site and may be adsorbed on the surface of the starch rather than bonded to the active site. Similarly, too much molecular crowding around the active site may prevent the odor molecule from reaching the metal, thus providing no increased effect over untreated expanded starch.

In any situation, after the bridging compound has been anchored to the expanded starch, and the metal binding site has been made available, the metal may then be joined with the binding site. After complexation, the metal may additionally have a number of innocuous ligands bonded to it. These could be in the form of the reaction solvent for metal treatment, water from the atmosphere, remaining ligands from the starting material, or the like. These residual ligands can be exchanged for a new set of specific ligands that in and of themselves, offer a unique scent. These specific scented ligands may displace the innocuous residual ligands on the metal center and may be displaced by a change in the overall system, such as complete hydration, associated with an insult event such as urination, incoming odorous molecules, or other triggers. Once released, the scent ligand volatilizes and provides a noticeable scent of its own.

As used herein, the term “residual ligand” refers to ligands joined during the derivation process and may include water for example. As used herein the term “specific ligands” refers to ligands selectively joined to the metal after the derivation process, wherein the specific ligands replace the residual ligands in a subsequent derivation step. Specific ligands may provide a pleasant scent, a masking scent, a complimentary scent, a training scent, or the like, or combinations thereof.

The ligands may have a triggered release wherein the bond with the metal is broken and the ligand is released. As used herein, a “triggered release” refers to ligands that are released from the metal by a molecule having a greater affinity for the metal than the given ligand. For example, an odorant may trigger the release of a ligand by overcoming the affinity the metal has for the given ligand. In other words, the affinity between the metal and the odor molecule may be greater than the affinity between the metal and the ligand. This is referred to herein as “odor-triggered release.” An example of an odor-triggered release includes a copper derivatized expanded starch particle with an alkene (e.g., limonene) ligand joined to the copper. When the derivatized expanded starch and ligand are exposed to an amine (e.g., triethylamine), it is expected that the ligand will be released and the amine will be captured because the affinity between the copper and the amine is greater than the affinity between the copper and the alkene. In another example, a water molecule may trigger the release of a ligand by overcoming the affinity the metal has for the given ligand. This is referred to herein as “water-triggered release.”

Any suitable scent molecule (any component of natural fragrance and/or synthetic perfume and/or odor) may be attached as a ligand. Examples of scent ligands include alcohols, ketones, aldehydes, esters, aromatics, terpenes, and the like. Specific examples include citronellol, benzaldehyde, alpha-pinene, 3-methylbutyl acetate, cymene, menthol, limonene, 2-butanone, and the like. The scented ligands may act to add a pleasant scent after activation or to mask an unpleasant odor present. They may also be used in combination with one another. The scented ligands may also be used to alert a caregiver that an insult is present by releasing the scented ligand when triggered by water and/or odor molecules. Additional suitable scent molecules are described in Chemistry of Fragrant Substances, by Paul Jose' Teisserie, VCH Publishers, Inc., New York, N.Y., 1994, and Perfumes, Cosmetics and Soaps, Volumes 1-3, by W. A. Poucher, Chapman and Hall, London, 1974.

The coordination complex of the present invention is believed to achieve high levels of odor reduction. For example, in some embodiments, the complex contains one or more free active sites capable of adsorbing an odorous compound. The complex, however, does not necessarily require the presence of free active sites. For example, one or more of the active sites may be occupied by a ligand bound weakly enough so that they are replaced by an odorous molecule when contacted therewith. Oxygen-based ligands, for instance, are normally weaker in their binding energies than nitrogen and sulfur ligands, and thus, may sometimes be replaced by an odorous molecule.

The expanded starch of the present invention may be derivatized by any suitable method as known to those skilled in the art. For example, one suitable method includes placing approximately 4 grams of expanded starch, prepared by the method described above, in a round bottom flask containing 35 ml of dry toluene and a stir bar. Next, 4.5 grams of 3-aminopropyltriethoxysilane may be slowly added with rapid stirring. The mixture may then be refluxed for 24 hours. After reflux, the mixture may then be allowed to cool and may be treated with an excess of ethanol resulting in an amino-derivatized expanded starch in solvent. The amino-derivatized expanded starch can then be vacuum filtered from the solvent and washed with more ethanol. The amino-derivatized expanded starch may then be further modified with 1.5 grams of 2-acetyl-pyridine in 100 ml of ethanol under reflux overnight to generate an immobilized Schiff base metal binding site. After reflux and after returning to room temperature, the immobilized Schiff base expanded starch may again be purified by vacuum filtration and washed with excess ethanol thereby resulting in a material that is ready to be treated with any suitable metal.

The metal atoms may be introduced by any suitable means. For example, copper(II) may be introduced into the metal binding site by stirring an aqueous ethanolic solution of the expanded starch with 175 mg of copper(II) chloride (CuCl₂). In another example, iron(III) may be introduced into the metal binding site by stirring an aqueous ethanolic solution of the expanded starch with 350 mg of ferric ammonium sulfate ((NH₄)Fe(SO₄)₂). In another example, manganese may be introduced into the metal binding site by stirring an aqueous ethanolic solution of the expanded starch with 164 mg of manganese(II) chloride salt (MnCl₂). In another example, zinc may be introduced into the metal binding site by stirring an aqueous ethanolic solution of the expanded starch with 177 mg of zinc(II) chloride salt (ZnCl₂). In another example, silver may be introduced into the metal binding site by stirring an aqueous ethanolic solution of the expanded starch with 222 mg of silver(I) nitrate (AgNO₃). Regardless of the metal used, the metal modified expanded starch may then be vacuum filtered from the solution and rinsed with an aqueous ethanol solution and dried thereby resulting in a metal derivatized expanded starch suitable for use as described herein.

An exemplary synthesis for producing metal derivatized expanded starch is illustrated below.

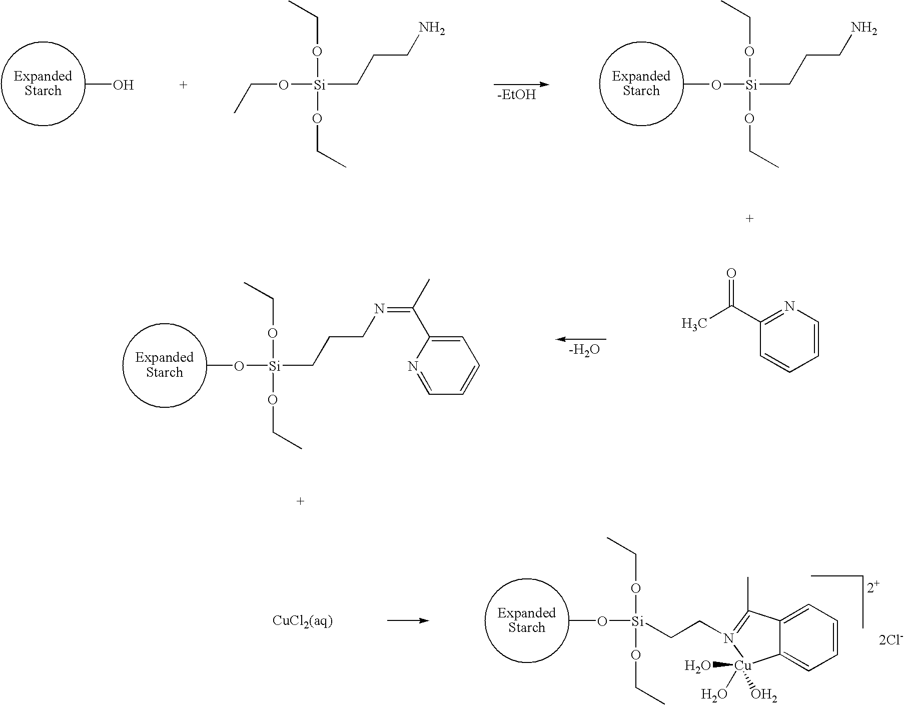

One skilled in the art will readily appreciate that many different combinations of expanded starches and derivatized expanded starches are conceivable. The following combinations are exemplary only.

In one embodiment, the expanded starch material may be modified as previously described utilizing 3-aminopropyltriethoxysilane and acetylpyridine to create a diimine-modified expanded starch material. This material may then be further modified with copper(II) chloride to generate a copper-bound expanded starch odor control material. This material may then be exposed to an alcoholic solution of limonene to replace the residual water ligands around the copper with limonene ligands. The resultant material of this embodiment may be utilized for odor control. The resultant material is expected to not only bind odor molecules generally from the environment, but is also expected to have a specific affinity for sulfur and amine odors as a result of the copper. It is believed that when an odor molecule is bound by the active copper center, the limonene ligand will be released thereby creating a pleasant, citrus-like fragrance (i.e., an odor-triggered release). In other words, the derivatized expanded starch of this embodiment is expected to control odors by at least three mechanisms. First, non-specific odor molecules may be absorbed on the surface of the expanded starch base. Second, specific odor molecules may be absorbed on the active sites of the copper. Third, the limonene ligands may be released to create a pleasant masking scent.

In another embodiment, the expanded starch material may be modified as previously described utilizing 3-aminopropyltriethoxysilane and acetylpyridine to create a diimine-modified expanded starch material. This material may then be further modified with ferric ammonium sulfate to generate an iron-bound expanded starch odor control material. This material may then be exposed to benzaldehyde to replace the residual water ligands around the iron with benzaldehyde ligands. The resultant material of this embodiment may be utilized for odor control. The resultant material is expected to not only bind odor molecules generally from the environment, but is also expected to have a specific affinity for amines as a result of the iron. It is believed that when an odor molecule is bound by the active iron center, the benzaldehyde ligand will be released thereby creating a pleasant, sweet cherry almond fragrance. In other words, the derivatized expanded starch of this embodiment is expected to control odors by at least three mechanisms as described previously.

In another embodiment, the expanded starch material may be modified as previously described utilizing 3-aminopropyltriethoxysilane and acetylpyridine to create a diimine-modified expanded starch material. The diimine-modified expanded starch material may then be treated with a lesser amount of copper(II) chloride resulting in copper-bound expanded starch particles. The copper-bound expanded starch particles may then be treated in a similar fashion with ferric ammonium sulfate to create a dimetallic expanded starch. It is believed that some of the expanded starch particle bases may include copper, some may include iron, and some may include both copper and iron. It is also expected that this material would exhibit an increased level of odor control activity, specifically around the control of amines. It is also expected that this dimetallic expanded starch would be extremely effective in controlling the odors associated with urine. The dimetallic expanded starch could further be treated with one or more pleasant smelling ligands, such as citronellol, that would be released upon ligand exchange when a more energetically favorable amine odor ligand binds to either metal site thereby replacing the less strongly bound citronellol scent ligand.

The expanded starches of the present invention (derivatized, non-derivatized, and combinations thereof) may be used alone for absorbing malodors or may be combined with various articles of manufacture. For example, the expanded starches of the present invention may be applied to a substrate. The substrate may provide an increased surface area to facilitate the adsorption of odorous compounds by the particles. In addition, the substrate may also serve other purposes, such as water absorption. Any of a variety of different substrates may be incorporated with the expanded starch particles in accordance with the present invention. For instance, nonwoven fabrics, woven fabrics, knit fabrics, wet-strength paper, film, foams, etc., may be applied with the expanded starch particles. When utilized, the nonwoven fabrics may include, but are not limited to, spunbonded webs (apertured or non-apertured), meltblown webs, bonded carded webs, air-laid webs, coform webs, hydraulically entangled webs, and so forth.

In some embodiments, for example, the expanded starch particles may be utilized in a paper product containing one or more paper webs, such as facial tissue, bath tissue, paper towels, napkins, and so forth. The paper product may be single-ply in which the web forming the product includes a single layer or is stratified (i.e., has multiple layers), or multi-ply, in which the webs forming the product may themselves be either single or multi-layered.

If desired, the substrate may form all or a portion of an absorbent article. In one embodiment, for instance, the absorbent article may include a liquid-pervious bodyside liner or “topsheet,” a liquid-pervious surge layer below the bodyside liner, a liquid-absorbent core below the surge layer, and a moisture vapor permeable, liquid impermeable outer cover or “backsheet” below the absorbent core. A substrate treated with the expanded starch particles of the present invention may be employed as any one or more of the liquid permeable (non-retentive) and absorbent layers. An absorbent core of the absorbent article, for instance, may be formed from an absorbent nonwoven web that includes a matrix of hydrophilic fibers. In one embodiment, the absorbent core may contain a matrix of cellulosic fluff fibers. In some embodiments, some or all of the expanded starch particles may be intermixed within the matrix of cellulose fluff fibers. In some embodiments, some or all of the expanded starch particles may be applied by any suitable means to one or more surfaces of the cellulose matrix.

Another type of suitable absorbent nonwoven web is a coform material, which is typically a blend of cellulose fibers and meltblown fibers. The term “coform” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and so forth. Some examples of such coform materials are disclosed in U.S. Pat. No. 4,100,324 to Anderson et al.; U.S. Pat. No. 5,284,703 to Everhart et al.; and U.S. Pat. No. 5,350,624 to Georger et al.; which are incorporated herein by reference where not contradictory. In some embodiments, some or all of the expanded starch particles may be intermixed within the coform absorbent material. In some embodiments, some or all of the expanded starch particles may be applied by any suitable means to one or more surfaces of the coform absorbent material.

As indicated above, the expanded starch particles may also be applied to a liquid transmissive layer of the absorbent article, such as the bodyside liner or surge layer. Such liquid transmissive layers are typically intended to transmit liquid quickly, and thus generally do not retain or absorb significant quantities of aqueous liquid. Materials that transmit liquid in such a manner include, but are not limited to, thermoplastic spunbonded webs, meltblown webs, bonded carded webs, air laid webs, and so forth. A wide variety of thermoplastic materials may be used to construct these non-retentive nonwoven webs, including without limitation polyamides, polyesters, polyolefins, copolymers of ethylene and propylene, copolymers of ethylene or propylene with a C₄-C₂₀ alpha-olefin, terpolymers of ethylene with propylene and a C₄-C₂₀ alpha-olefin, ethylene vinyl acetate copolymers, propylene vinyl acetate copolymers, styrene-poly(ethylene-alpha-olefin) elastomers, polyurethanes, A-B block copolymers where A is formed of poly(vinyl arene) moieties such as polystyrene and B is an elastomeric midblock such as a conjugated diene or lower alkene, polyethers, polyether esters, polyacrylates, ethylene alkyl acrylates, polyisobutylene, poly-1-butene, copolymers of poly-1-butene including ethylene-1-butene copolymers, polybutadiene, isobutylene-isoprene copolymers, and combinations of any of the foregoing.

The expanded starch particles may be applied to a substrate using any of a variety of well-known application techniques. Suitable techniques for applying the composition to a substrate include printing, dipping, spraying, melt extruding, solvent coating, powder coating, and so forth. The expanded starch particles may be incorporated within the matrix of the substrate and/or applied to at least one surface thereof.

In one embodiment, an absorbent article may include a liquid permeable bodyside liner, a liquid impermeable outer cover, and an absorbent core located between the liner and the outer cover. The absorbent core may include expanded starch particles in any suitable configuration. For example, the absorbent core may include absorbent fibers, such as cellulose fluff fibers and expanded starch particles. The absorbent core may include strata of absorbent materials wherein the expanded starch particles are located generally within a layer above, below, or between one or more fluff layers. Alternatively, the absorbent core may include expanded starch particles partially or completely intermixed with the absorbent fibers. The expanded starch particles may be present in any suitable concentration and any suitable location. For example, the expanded starch particles may be located in the front portion and/or back portion and/or the edges of the absorbent product to minimize contact with fluids within the article. In another example, a substrate treated with the expanded starch particles of the present invention may be employed as any one or more of the liquid transmissive (non-retentive) and absorbent layers.

In some embodiments, the expanded starch particles may be positioned so as to avoid immediate contact by body fluids discharged by the user. As discussed previously, the expanded starch is most effective when dry, but still remains effective when wet. Therefore, the expanded starch may be positioned within the absorbent article so as to intersect vapors emanating from the article and thereby absorb the malodors. In this regard, the expanded starch may be located around the peripheral edge of the absorbent article near the lateral sides and/or the longitudinal ends. In these positions, the expanded starch is expected to remain dry until the absorbent article has absorbed a significant amount of fluid relative to its ultimate capacity.

In other embodiments, the expanded starch particles may be positioned in a central portion of the product, but shielded by hydrophobic fibers in order to minimize its contact by body fluid while still allowing it to absorb malodors. The expanded starch can further be placed within a fibrous material that is hydrophobic in order to discourage passage of fluid therethrough (i.e., an “enclosure”). For example, the absorbent article may include a vapor permeable member or layer positioned between the absorbent and the liquid-impermeable baffle. The vapor permeable member may be a nonwoven, fibrous web which is preferably liquid-impermeable. The vapor permeable member may be bonded to the absorbent, the outer cover, or both by any suitable means. The expanded starch particles may then be positioned between the vapor permeable member and the liquid-impermeable outer cover. The vapor permeable member is expected to allow the malodors from the absorbent to emanate therethrough and be absorbed by the expanded starch while providing a barrier to minimize the wetting of the expanded starch.

The expanded starch particles of the present invention may also be used with other types of articles of manufacture. For instance, the expanded starch particles may be used in air filters, such as house filters, vent filters, disposable facemasks and facemask filters. Additionally, the expanded starch particles may be applied to walls, wallpaper, glass, toilets and/or countertops. For instance, the expanded starch particles may be used in a restroom facility, or as pet litter. Other uses include, without limitation, refrigerator mats, dryer sheets and fabric softener sheets.

In various embodiments, any suitable combination of one or more types of expanded starches and/or one or more types of derivatized expanded starches may be included in any suitable article, such as a diaper. As used herein the term “types of expanded starches” refers to the different native starches from which expanded starches are derived. For example, corn, potato, rice, etc., are different types of expanded starches. Also as used herein, the term “types of derivatized expanded starches” refers to the different metals joined with the expanded starch bases. For example, iron, zinc, copper, etc., are different types of metal derivatized expanded starches. In some embodiments, different types of expanded starches may be derivatized with the same metal. For example, both expanded corn starch and expanded rice starch may be derivatized with iron.

While the invention has been described in detail with respect to specific embodiments thereof, it will be appreciated that those skilled in the art, upon attaining understanding of the foregoing will readily appreciate alterations to, variations of, and equivalents to these embodiments. Accordingly, the scope of the present invention should be assessed as that of the appended claims and any equivalents thereto.

1. An absorbent article comprising a plurality of derivatized expanded starch particles wherein the derivatized expanded starch particles comprise an expanded starch particle base and at least one transition metal chemically bonded to the expanded starch particle base.

2. The absorbent article of claim 1 wherein the transition metal is copper, iron, manganese, zinc or silver.

3. The absorbent article of claim 2 wherein the transition metal comprises at least one ligand chemically bonded thereto, wherein the ligand is citronellol, benzaldehyde, alpha-pinene, 3-methylbutyl acetate, cymene, menthol, limonene or 2-butanone.

4. The absorbent article of claim 1 wherein the transition metal is coordinatively bonded to a bridging compound and the bridging compound is covalently bonded to a surface of the expanded starch particle bases.

5. The absorbent article of claim 4 wherein the bridging compound comprises a siloxane anchoring group covalently bonded to a metal binding site.

6. The absorbent article of claim 1 wherein the transition metal is coordinatively bonded to a bridging compound and the bridging compound is physically absorbed to a surface of the expanded starch particle bases.

7. The absorbent article of claim 6 wherein the bridging compound comprises an ionic anchoring group covalently bonded to a metal binding site, wherein the ionic anchoring group is selected from the group consisting of quarternary ammonium, carboxylate, sulfonate, phosphate, sulfate and phosphonate.

8. The absorbent article of claim 1 wherein at least one first derivatized expanded starch particle comprises,
a first expanded starch particle base,
a first transition metal chemically bonded to the first expanded starch particle base, and
a second transition metal chemically bonded to the first expanded starch particle base, wherein the first transition metal and the second transition metal are different.

9. The absorbent article of claim 1 wherein at least one first derivatized expanded starch particle comprises a first expanded starch particle base having a first transition metal chemically bonded to the first expanded starch particle base and at least one second derivatized expanded starch particle comprises a second expanded starch particle base having a second transition metal chemically bonded to the second expanded starch particle base, wherein the first and second transition metals are different.

10. The absorbent article of claim 9 wherein the first transition metal is copper and the second transition metal is iron.

11. An absorbent article comprising at least one nonderivatized expanded starch particle and at least one derivatized expanded starch particle, wherein the at least one derivatized expanded starch particle comprises an expanded starch particle base and one or more transition metals chemically bonded to the expanded starch particle base.

12. The absorbent article of claim 11 wherein the transition metal is copper, iron, manganese, zinc or silver.

13. The absorbent article of claim 12 wherein the transition metal comprises at least one ligand chemically bonded thereto, wherein the ligand is citronellol, benzaldehyde, alpha-pinene, 3-methylbutyl acetate, cymene, menthol, limonene or 2-butanone.

14. The absorbent article of claim 13 wherein the transition metal is chemically bonded to a bridging compound and the bridging compound is covalently bonded to a surface of the expanded starch particle bases.

15. The absorbent article of claim 11 wherein
at least one first derivatized expanded starch particle comprises,
a first expanded starch particle base,
a first transition metal chemically bonded to the first expanded starch particle base, and
a second transition metal chemically bonded to the first expanded starch particle base, wherein the first transition metal and the second transition metal are different.

16. The absorbent article of claim 11 wherein at least one first derivatized expanded starch particle comprises a first expanded starch particle base having a first transition metal chemically bonded to the first expanded starch particle base and at least one second derivatized expanded starch particle comprises a second expanded starch particle base having a second transition metal chemically bonded to the second expanded starch particle base, wherein the first and second transition metals are different.

17. An absorbent article comprising a plurality of derivatized expanded starch particles, a first transition metal, and a second transition metal, wherein the first and second transition metals are different.

18. The absorbent article of claim 17 wherein the derivatized expanded starch particles comprise an expanded starch particle base and the first transition metal and the second transition metal are chemically bonded to bridging compounds and the bridging compounds are covalently bonded to a surface of the expanded starch particle bases.

19. The absorbent article of claim 18 wherein at least one first derivatized expanded starch particle comprises,
a first expanded starch particle base,
the first transition metal chemically bonded to the first expanded starch particle base, and
the second transition metal chemically bonded to the first expanded starch particle base.

20. The absorbent article of claim 18 wherein at least one first derivatized expanded starch particle comprises a first expanded starch particle base having the first transition metal chemically bonded to the first expanded starch particle base and at least one second derivatized expanded starch particle comprises a second expanded starch particle base having the second transition metal chemically bonded to the second expanded starch particle base.
